SEQUENCE LISTING (1 sequence)

SEQ ID NO: 1 - protein, 1232 aa, synthetic construct
MLHAFTNQYQLSKTLRFGATLKEDEKKCKSHEELKGFVDISYENMKSSATIAESLNENELVKKCERCYSEIVKFHNAWEKIYYRTDQIAVYKDFYRQLSRKARFDAGKQNSQLITLASLCGMYQGAKLSRYITNYWKDNITRQKSFLKDFSQQLHQYTRALEKSDKAHTKPNLINFNKTFMVLANLVNEIVIPLSNGAISFPNISKLEDGEESHLIEFALNDYSQLSELIGELKDAIATNGGYTPFAKVTLNHYTAEQKPHVFKNDIDAKIRELKLIGLVETLKGKSSEQIEEYFSNLDKFSTYNDRNQSVIVRTQCFKYKPIPFLVKHQLAKYISEPNGWDEDAVAKVLDAVGAIRSPAHDYANNQEGFDLNHYPIKVAFDYAWEQLANSLYTTVTFPQEMCEKYLNSIYGCEVSKEPVFKFYADLLYIRKNLAVLEHKNNLPSNQEEFICKINNTFENIVLPYKISQFETYKKDILAWINDGHDHKKYTDAKQQLGFIRGGLKGRIKAEEVSQKDKYGKIKSYYENPYTKLTNEFKQISSTYGKTFAELRDKFKEKNEITKITHFGIIIEDKNRDRYLLASELKHEQINHVSTILNKLDKSSEFITYQVKSLTSKTLIKLIKNHTTKKGAISPYADFHTSKTGFNKNEIEKNWDNYKREQVLVEYVKDCLTDSTMAKNQNWAEFGWNFEKCNSYEDIEHEIDQKSYLLQSDTISKQSIASLVEGGCLLLPIINQDITSKERKDKNQFSKDWNHIFEGSKEFRLHPEFAVSYRTPIEGYPVQKRYGRLQFVCAFNAHIVPQNGEFINLKKQIENFNDEDVQKRNVTEFNKKVNHALSDKEYVVIGIDRGLKQLATLCVLDKRGKILGDFEIYKKEFVRAEKRSESHWEHTQAETRHILDLSNLRVETTIEGKKVLVDQSLTLVKKNRDTPDEEATEENKQKIKLKQLSYIRKLQHKMQTNEQDVLDLINNEPSDEEFKKRIEGLISSFGEGQKYADLPINTMREMISDLQGVIARGNNQTEKNKIIELDAADNLKQGIVANMIGIVNYIFAKYSYKAYISLEDLSRAYGGAKSGYDGRYLPSTSQDEDVDFKEQQNQMLAGLGTYQFFEMQLLKKLQKIQSDNTVLRFVPAFRSADNYRNILRLEETKYKSKPFGVVHFIDPKFTSKKCPVCSKTNVYRDKDDILVCKECGFRSDSQLKERENNIHYIHNGDDNGAYHIALKSVENLIQMK

(19) United States
(12) Patent Application Publication
(10) Pub. No.: US 2025/0354130 A1
(43) Pub. Date: Nov. 20, 2025

(54) COMPOSITIONS AND METHODS RELATED TO MODIFIED CAS12A2 MOLECULES

(71) Applicants: BOARD OF REGENTS, THE UNIVERSITY OF TEXAS SYSTEM, Austin, TX (US); UTAH STATE UNIVERSITY, Logan, UT (US)

(72) Inventors: David TAYLOR, Austin, TX (US); Jack P.K. BRAVO, Austin, TX (US); Ryan JACKSON, Logan, UT (US); Thomson J. HALLMARK, Logan, UT (US)

(21) Appl. No.: 18/872,146

(22) PCT Filed: Jun. 6, 2023
(86) PCT No.: PCT/US2023/067968

§ 371 Date: Dec. 5, 2024

Related U.S. Application Data
(60) Provisional application No. 63/349,225, filed on Jun. 6, 2022.
(60) Provisional application No. 63/385,260, filed on Nov. 29, 2022.

(51) Int. Cl. C12N 9/22

(52) U.S. Cl. CPC C12N 9/226

(57) ABSTRACT

RNA-targeting Cas12a2 complex allows for rationale design of Cas12a2 into a versatile enzyme capable of non-specifically degrading distinct types of nucleic acid targets depending on mutations of the active site residues and residues that stabilize bound targets. These mutations allow for tuning of output signal associated with RNA detection. By mutating specific residues, indiscriminate single-stranded RNase and DNase and double-stranded DNase activity can be modified to only cleave single-stranded DNA and single-stranded RNA, or only single-stranded DNA. This allows for diagnostic tools which can provide a detection. Residues involved in binding the non-self vs. self-recognition signal (PFS) can also be modified so larger subsets of nucleic acid targets can be recognized.

### CROSS-REFERENCE TO RELATED APPLICATIONS

This application claims benefit of U.S. Provisional Application No. 63/349,225, filed Jun. 6, 2022, and U.S. Provisional Application No. 63/385,260, filed Nov. 29, 2022, both of which are hereby incorporated herein by reference in their entirety.

### GOVERNMENT SUPPORT CLAUSE

This invention was made with government support under Grant no. R35 GM138080 and Grant No. R35 GM138348 awarded by the National Institutes of Health. The government has certain rights in the invention.

### REFERENCE TO SEQUENCE LISTING

The Sequence Listing submitted herewith as a text file named “10046-484WO1.XML”, created on Jun. 6, 2023, and having a size of 3223 bytes, is hereby incorporated by reference pursuant to 37 C.F.R. § 1.52(e)(5).

### BACKGROUND

Prokaryotic adaptive immunity typically utilizes CRISPR-Cas systems to target and degrade mobile genetic elements (MGE, including phage, transposons and plasmids) (Hampton et al., 2020; Makarova et al., 2020). However, it was recently discovered that Cas12a2 from Sulfuricurvum sp. PC08-66 instead relies on abortive infection—that is, bacterial suicide in response to the presence of an invader—to achieve population-level immunity via sacrifice of infected cells to prevent the replication and transmission of MGEs (Dmytrenko 2022).

Cas12a2 co-occurs with Cas12a systems in bacteria and can utilize Cas12a crRNA, Cas12a2 recognizes an RNA target strand with a suitable protospacer-flanking sequence (PFS) rather than the double-stranded (ds)DNA target of Cas12a (Dmytrenko 2022). Furthermore, Cas12a2 is immune to the effects of many anti-CRISPR (Acr) proteins that target Cas12a, and aside from a conserved RuvC nuclease domain, Cas12a and Cas12a2 sequences bear little resemblance to one another (˜10-20%). Notably, Cas12a2 lacks a Nuc domain (involved in DNA target strand loading), but instead contains a zinc-ribbon and a unique insertion domain.

The cell killing activity of Cas12a2 is mediated by robust, nonspecific cleavage of single-stranded (ss)RNA, ssDNA, and dsDNA unleashed by recognition of a target RNA. While other type V CRISPR systems elicit non-specific single-stranded nucleic acid degradation in trans upon activation (Chen et al., 2018; Yan et al., 2019), the collateral degradation of dsDNA stands is unique to Cas12a2, suggesting a distinct mechanism of activation. However, the molecular basis of duplex degradation in trans by Cas12a2 remains enigmatic.

What is needed in the art are variant Cas12a2 molecules and methods of using them.

### BRIEF DESCRIPTION OF THE FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1A-D: Cas12a2 binary complex resembles an oyster and orders the crRNA. a, Domain organization of Cas12a2. b, Cryo-EM structure of Cas12a2 binary complex colored by structural domain as in (a). c, Atomic model of Cas12a2 binary complex. d, Putative 3′ seed region of crRNA. Seven bases from the 3′ end are ordered, and bases are solvent-exposed, likely acting as a seed for TS RNA binding.

FIG. 2A-G: Target binding leads to large-scale structural arrangements for activation. a, Schematic of crRNA:TS duplex. Protospacer-flanking sequence (PFS) on TS is highlighted. b, Cryo-EM structure of Cas12a2 ternary complex. c, Atomic model of Cas12a2 ternary complex. d, Motion vector map showing conformational changes of Cas12a2 induced upon ternary complex formation. Binary complex model shown as grey cartoon. e, Surface electrostatic potential of binary and ternary complex, showing how active site becomes exposed upon ternary complex formation. This is accompanied by the formation of a large positively-charged groove adjacent to the activate site. f, Displacement of RuvC gating helix by ˜8 Å upon ternary complex formation exposes active site residues. g, target protection assay, showing cleavage when target is in molar excess and protection when binary complex is in excess. Protection persists for 2H.

FIG. 3A-H Cas12a2 binds and clamps duplex DNA. a, Cryo-EM structure of Cas12a2 quaternary complex. Trans-dsDNA is shown. b, Atomic model of Cas12a2 quaternary complex. c, dsDNA situated within active site. d, Close-up view of Cas12a2 active site e, Cleaved strand held in place through aromatic clamp. f, Non-cleaved strand held in place through aromatic clamp. g, Schematic of interactions between Cas12a2 and the collateral dsDNA substrate h, Cleavage of ssRNA (top), ssDNA (middle) and dsDNA (bottom) by Cas12a2, and aromatic clamp mutants.

FIG. 4A-O shows Cryo-EM quality control.

FIG. 5A-H shows comparison of Cas12a2 and Cas12a.

FIG. 6A-D shows PFS recognition. a) 5′-GAAAG-3′ PFS motif at 3′ end of TS (orange). b) Position of PFS within binary complex. Positions 1-4 are shown in boxes. c) Close-up view of positions 1-4 of PFS as denoted in panel b. Position (G)5 is flexible and does not participate in interactions with Cas12a2. d) Nucleic acid cleavage relies on PI recognition of PFS.

FIG. 7A-B shows conformational changes of insertion domain.

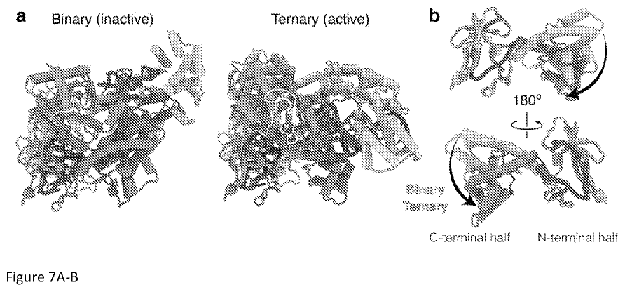

FIG. 8A-E shows representative EM density for active site, ZR, other regions of interest.

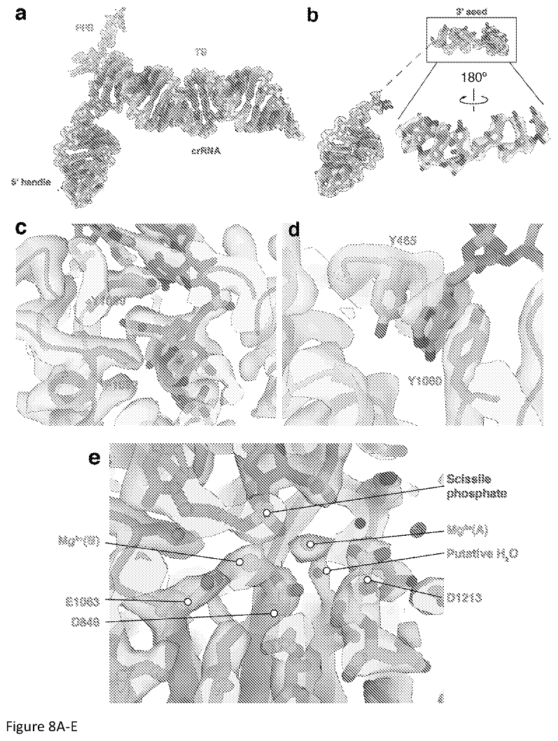

FIG. 9A-F shows structural basis for Anti-CRISPR evasion by Cas12a2. Top—Cas12a binary complex (PDB 5NG6, 9A) and Cas12a2 (9B) with AcrVA1 (PDB 6NMD). AcrVA1 interacts with the 5′ crRNA seed in Cas12a, which is not present in Cas12a2. There are also significant clashes between AcrVA1 and the REC lobe of Cas12a2. Middle—Cas12a (9C) and Cas12a2 (9D) binary complex with a monomer of AcrVA4 (PDB 6NMA). There are severe clashes between the Cas12a2 Insertion domain and AcrVA4. Bottom—PI domain of Cas12a (9E), showing recognition of the PAM sequence by K671. This is the equivalent residue targeted for acetylation by AcrVA5. For Cas12a2 (9F), there are multiple lysine residues that interact with the TS PFS. Since these residues interact with a single-stranded PFS rather than a double-stranded PAM, acetylation is unlikely to result in a steric clash that prevents stable association of a nucleic acid target with Cas12a2.

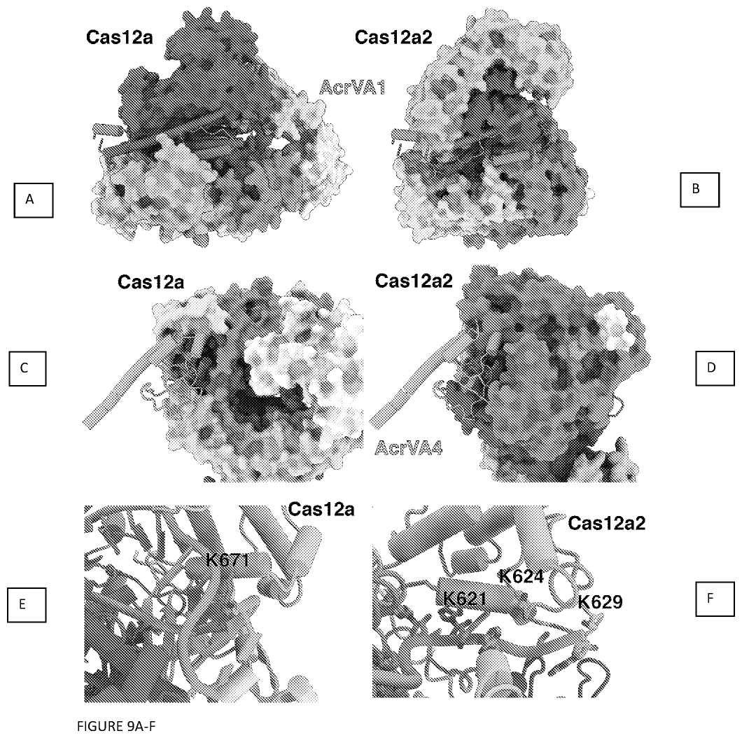

FIG. 10A-B shows that RNA inhibits Cas12a2 Y1069A. (A) shows inhibition of Cas12a2 Y1069A by RNA as a function of fluorescence over time. (B) shows a schematic of inhibition by RNA.

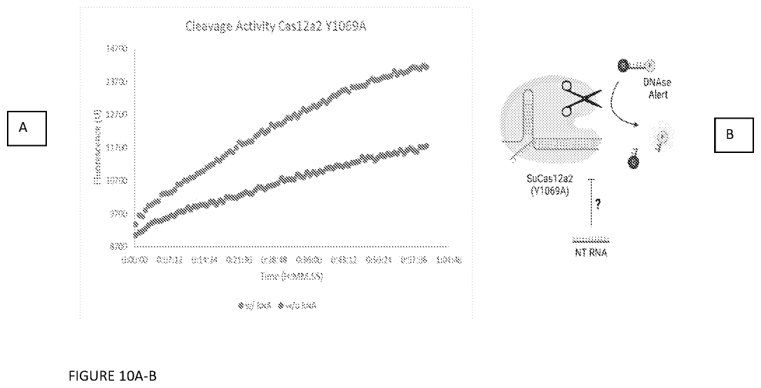

### DETAILED DESCRIPTION

### General Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence”, “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria, or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region.

“Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800. 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) o/Cell Biol 4:2253-8; Ayares et al., (1986) Proc. Natl. Acad. Sci. USA 83:5199-203; Liskay et al., (1987) Genetics 115: 161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8: 189-191) and found in the MegAlign™ program of the LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison, WI). Default parameters for multiple alignment (GAP PENALTY=10, GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters:% identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89: 10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps, using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”, “substantially similar” and“corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques, or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified organism. Genes can be designed for use in suppression by linking a nucleic acid fragment, whether or not it encodes an active enzyme, in the sense or antisense orientation relative to a native promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that organism is homozygous at that locus. If the alleles present at a given locus on a chromosome differ, that organism is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention. Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated organism is an organism comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR), wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

The term “conserved domain” or “motif” means a set of polynucleotides or amino acids conserved at specific positions along an aligned sequence of evolutionarily related proteins. While amino acids at other positions can vary between homologous proteins, amino acids that are highly conserved at specific positions indicate amino acids that are essential to the structure, the stability, or the activity of a protein. Because they are identified by their high degree of conservation in aligned sequences of a family of protein homologues, they can be used as identifiers, or “signatures”, to determine if a protein with a newly determined sequence belongs to a previously identified protein family.

A “codon-modified gene” or “codon-preferred gene” or “codon-optimized gene” is a gene having its frequency of codon usage designed to mimic the frequency of preferred codon usage of the host cell.

An “optimized” polynucleotide is a sequence that has been optimized for improved expression in a particular heterologous host cell.

A “promoter” is a region of DNA involved in recognition and binding of RNA polymerase and other proteins to initiate transcription. The promoter sequence consists of proximal and more distal upstream elements, the latter elements often referred to as enhancers.

An “enhancer” is a DNA sequence that can stimulate promoter activity and may be an innate element of the promoter or a heterologous element inserted to enhance the level or tissue-specificity of a promoter. Promoters may be derived in their entirety from a native gene or be composed of different elements derived from different promoters found in nature, and/or comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity.

Promoters that cause a gene to be expressed in most cell types at most times are commonly referred to as “constitutive promoters”. The term “inducible promoter” refers to a promoter that selectively express a coding sequence or functional RNA in response to the presence of an endogenous or exogenous stimulus, for example by chemical compounds (chemical inducers) or in response to environmental, hormonal, chemical, and/or developmental signals. Inducible or regulated promoters include, for example, promoters induced or regulated by light, heat, stress, flooding or drought, salt stress, osmotic stress, phytohormones, wounding, or chemicals such as ethanol, abscisic acid (ABA), jasmonate, salicylic acid, or safeners.

“Translation leader sequence” refers to a polynucleotide sequence located between the promoter sequence of a gene and the coding sequence. The translation leader sequence is present in the mRNA upstream of the translation start sequence. The translation leader sequence may affect processing of the primary transcript to mRNA, mRNA stability or translation efficiency. Examples of translation leader sequences have been described (e.g., Turner and Foster, (1995) Mol Biotechnol 3:225-236).

“3′ non-coding sequences”, “transcription terminator” or “termination sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complimentary copy of the DNA sequence, it is referred to as the primary transcript or pre-mRNA. An RNA transcript is referred to as the mature RNA or mRNA when it is a RNA sequence derived from post-transcriptional processing of the primary transcript pre-mRNA. “Messenger RNA” or “mRNA” refers to the RNA that is without introns and that can be translated into protein by the cell.

“cDNA” refers to a DNA that is complementary to, and synthesized from, an mRNA template using the enzyme reverse transcriptase. The cDNA can be single-stranded or converted into double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (see, e.g., U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “genome” refers to the entire complement of genetic material (genes and non-coding sequences) that is present in each cell of an organism, or virus or organelle; and/or a complete set of chromosomes inherited as a (haploid) unit from one parent.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., the coding sequence is under the transcriptional control of the promoter). Coding sequences can be operably linked to regulatory sequences in a sense or antisense orientation. In another example, the complementary RNA regions can be operably linked, either directly or indirectly, 5′ to the target mRNA, or 3′ to the target mRNA, or within the target mRNA, or a first complementary region is 5′ and its complement is 3′ to the target mRNA.

Generally, “host” refers to an organism or cell into which a heterologous component (polynucleotide, polypeptide, other molecule, cell) has been introduced. As used herein, a “host cell” refers to an in vivo or in vitro eukaryotic cell, prokaryotic cell (e.g., bacterial or archaeal cell), or cell from a multicellular organism (e.g., a cell line) cultured as a unicellular entity, into which a heterologous polynucleotide or polypeptide has been introduced. In some embodiments, the cell is selected from the group consisting of: an archaeal cell, a bacterial cell, a eukaryotic cell, a eukaryotic single-cell organism, a somatic cell, a germ cell, a stem cell, a plant cell, an algal cell, an animal cell, in invertebrate cell, a vertebrate cell, a fish cell, a frog cell, a bird cell, an insect cell, a mammalian cell, a pig cell, a cow cell, a goat cell, a sheep cell, a rodent cell, a rat cell, a mouse cell, a non-human primate cell, and a human cell. In some cases, the cell is in vitro. In some cases, the cell is in vivo.

The term “recombinant” refers to an artificial combination of two otherwise separated segments of sequence, e.g., by chemical synthesis, or manipulation of isolated segments of nucleic acids by genetic engineering techniques.

The terms “plasmid”, “vector” and “cassette” refer to a linear or circular extra chromosomal element often carrying genes that are not part of the central metabolism of the cell, and usually in the form of double-stranded DNA. Such elements may be autonomously replicating sequences, genome integrating sequences, phage, or nucleotide sequences, in linear or circular form, of a single- or double-stranded DNA or RNA, derived from any source, in which a number of nucleotide sequences have been joined or recombined into a unique construction which is capable of introducing a polynucleotide of interest into a cell.

“Transformation cassette” refers to a specific vector comprising a gene and having elements in addition to the gene that facilitates transformation of a particular host cell. “Expression cassette” refers to a specific vector comprising a gene and having elements in addition to the gene that allow for expression of that gene in a host.

The terms “recombinant DNA molecule”, “recombinant DNA construct”, “expression construct”, “construct”, and “recombinant construct” are used interchangeably herein. A recombinant DNA construct comprises an artificial combination of nucleic acid sequences, e.g., regulatory and coding sequences that are not all found together in nature. For example, a recombinant DNA construct may comprise regulatory sequences and coding sequences that are derived from different sources, or regulatory sequences and coding sequences derived from the same source but arranged in a manner different than that found in nature. Such a construct may be used by itself or may be used in conjunction with a vector. If a vector is used, then the choice of vector is dependent upon the method that will be used to introduce the vector into the host cells as is well known to those skilled in the art. For example, a plasmid vector can be used. The skilled artisan is well aware of the genetic elements that must be present on the vector in order to successfully transform, select and propagate host cells. The skilled artisan will also recognize that different independent transformation events may result in different levels and patterns of expression (Jones et al., (1985) EMBO J 4:2411-2418; De Almeida et al., (1989) Mol Gen Genetics 218:78-86), and thus that multiple events are typically screened in order to obtain lines displaying the desired expression level and pattern. Such screening may be accomplished standard molecular biological, biochemical, and other assays including Southern analysis of DNA, Northern analysis of mRNA expression, PCR, real time quantitative PCR (qPCR), reverse transcription PCR (RT-PCR), immunoblotting analysis of protein expression, enzyme or activity assays, and/or phenotypic analysis.

The term “heterologous” refers to the difference between the original environment, location, or composition of a particular polynucleotide or polypeptide sequence and its current environment, location, or composition. As used herein, “heterologous” in reference to a sequence can refer to a sequence that originates from a different species, variety, foreign species, or, if from the same species, is substantially modified from its native form in composition and/or genomic locus by deliberate human intervention. For example, a promoter operably linked to a heterologous polynucleotide is from a species different from the species from which the polynucleotide was derived, or, if from the same/analogous species, one or both are substantially modified from their original form and/or genomic locus, or the promoter is not the native promoter for the operably linked polynucleotide. Alternatively, one or more regulatory region(s) and/or a polynucleotide provided herein may be entirely synthetic. In another example, a target polynucleotide for cleavage by a Cas endonuclease may be of a different organism than that of the Cas endonuclease. In another example, a Cas endonuclease and guide RNA may be introduced to a target polynucleotide with an additional polynucleotide that acts as a template or donor for insertion into the target polynucleotide, wherein the additional polynucleotide is heterologous to the target polynucleotide and/or the Cas endonuclease.

The term “expression”, as used herein, refers to the production of a functional end-product (e.g., an mRNA, guide RNA, or a protein) in either precursor or mature form.

A “mature” protein refers to a post-translationally processed polypeptide (i.e., one from which any pre- or propeptides present in the primary translation product have been removed). “Precursor” protein refers to the primary product of translation of mRNA (i.e., with pre- and propeptides still present). Pre- and propeptides may be but are not limited to intracellular localization signals.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array, comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules that comprise a Rec3 clamp, as described below.

As used herein, the term “Cas12a2 protein” (also referred to as “Cpf1”) refers to, but is not limited to, Cas12a2 proteins, Cas12a2-type proteins encoded by Cas12a2 orthologs, and synthetic proteins of Cas12a2. The term “Cas12a2 protein” as used herein refers to a wild type Cas12a2 protein from CRISPR-Cas12a2 systems, Cas12a2 protein modifications, Cas12a2 protein variants, Cas12a2 orthologs and combinations of the same. The Cas12a2 molecule is represented herein by SEQ ID NO: 1.

A Cas protein is further defined as a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450, at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein, and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains.

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′, or any combination thereof) to those domains typical of a Cas endonuclease.

The terms “Cascade” and “Cascade complex” are used interchangeably herein and include reference to a multi-subunit protein complex that can assemble with a polynucleotide forming a polynucleotide-protein complex (PNP). Cascade is a PNP that relies on the polynucleotide for complex assembly and stability, and for the identification of target nucleic acid sequences. Cascade functions as a surveillance complex that finds and optionally binds target nucleic acids that are complementary to a variable targeting domain of the guide polynucleotide.

The terms “cleavage-ready Cascade”, “crCascade”, “cleavage-ready Cascade complex”, “crCascade complex”, “cleavage-ready Cascade system”, “CRC” and “crCascade system”, are used interchangeably herein and include reference to a multi-subunit protein complex that can assemble with a polynucleotide forming a polynucleotide-protein complex (PNP), wherein one of the cascade proteins is a Cas endonuclease capable of recognizing, binding to, and optionally unwinding, nicking, or cleaving all or part of a target sequence.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be a RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA).

The term “variable targeting domain” or “VT domain” is used interchangeably herein and includes a nucleotide sequence that can hybridize (is complementary) to one strand (nucleotide sequence) of a double strand DNA target site. The percent complementation between the first nucleotide sequence domain (VT domain) and the target sequence can be at least 50%, 51%, 52%, 53%, 54%, 55%, 56%, 57% 58%, 59% 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable targeting domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example E1S20150059010A1, published 26 Feb. 2015), or any combination thereof.

As used herein, the terms “guide polynucleotide/Cas endonuclease complex”, “guide polynucleotide/Cas endonuclease system”, “guide polynucleotide/Cas complex”, “guide polynucleotide/Cas system” and “guided Cas system,” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327: 167-170; Makarova et al. 2015, Nature Reviews Microbiology Vol. 13: 1-15; Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”, “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The term “transposon”, as used herein, refers to a polynucleotide (or nucleic acid segment), which may be recognized by a transposase or an integrase enzyme and which is a component of a functional nucleic acid-protein complex (e.g., a transpososome) capable of transposition. The term “transposase” as used herein refers to an enzyme, which is a component of a functional nucleic acid-protein complex capable of transposition and which mediates transposition. The transposase may comprise a single protein or comprise multiple protein sub-units. A transposase may be an enzyme capable of forming a functional complex with a transposon end or transposon end sequences. The term “transposase” may also refer in certain embodiments to integrases. The expression “transposition reaction” used herein refers to a reaction wherein a transposase inserts a donor polynucleotide sequence in or adjacent to an insertion site on a target polynucleotide. The insertion site may contain a sequence or secondary structure recognized by the transposase and/or an insertion motif sequence where the transposase cuts or creates staggered breaks in the target polynucleotide into which the donor polynucleotide sequence may be inserted. Exemplary components in a transposition reaction include a transposon, comprising the donor polynucleotide sequence to be inserted, and a transposase or an integrase enzyme. The term, “transposon end sequence” as used herein refers to the nucleotide sequences at the distal ends of a transposon. The transposon end sequences may be responsible for identifying the donor polynucleotide for transposition. The transposon end sequences may be the DNA sequences the transpose enzyme uses in order to form transpososome complex and to perform a transposition reaction.

The terms “target site”, “target sequence”, “target site sequence,” target DNA”, “target locus”, “genomic target site”, “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

As used herein, terms “endogenous target sequence” and “native target sequence” are used interchangeable herein to refer to a target sequence that is endogenous or native to the genome of a cell and is at the endogenous or native position of that target sequence in the genome of the cell. An “artificial target site” or “artificial target sequence” are used interchangeably herein and refer to a target sequence that has been introduced into the genome of a cell. Such an artificial target sequence can be identical in sequence to an endogenous or native target sequence in the genome of a cell but be located in a different position (i.e., a non-endogenous or non-native position) in the genome of a cell.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19 or 20 nucleotides long.

An “altered target site”, “altered target sequence”, “modified target site”, “modified target sequence” are used interchangeably herein and refer to a target sequence as disclosed herein that comprises at least one alteration when compared to non-altered target sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

A “modified nucleotide” or “edited nucleotide” refers to a nucleotide sequence of interest that comprises at least one alteration when compared to its non-modified nucleotide sequence. Such “alterations” include, for example: (i) replacement of at least one nucleotide, (ii) a deletion of at least one nucleotide, (iii) an insertion of at least one nucleotide, (iv) a chemical alteration of at least one nucleotide, or (v) any combination of (i)-(iv).

Methods for “modifying a target site” and “altering a target site” are used interchangeably herein and refer to methods for producing an altered target site.

As used herein, “donor DNA” is a DNA construct that comprises a polynucleotide of interest to be inserted into the target site of a Cas endonuclease.

The term “polynucleotide modification template” includes a polynucleotide that comprises at least one nucleotide modification when compared to the nucleotide sequence to be edited. A nucleotide modification can be at least one nucleotide substitution, addition, or deletion. Optionally, the polynucleotide modification template can further comprise homologous nucleotide sequences flanking the at least one nucleotide modification, wherein the flanking homologous nucleotide sequences provide sufficient homology to the desired nucleotide sequence to be edited.

A “complex trait locus” includes a genomic locus that has multiple transgenes genetically linked to each other.

The terms “decreased,” “fewer,” “slower” and “increased” “faster” “enhanced” “greater” as used herein refers to a decrease or increase in a property such as efficiency, processivity, or specificity. For example, a decrease in a characteristic may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400%) or more lower than the wild type or other control, and an increase may be at least 1%, at least 2%, at least 3%, at least 4%, at least 5%, between 5% and 10%, at least 10%, between 10% and 20%, at least 15%, at least 20%, between 20% and 30%, at least 25%, at least 30%, between 30% and 40%, at least 35%, at least 40%, between 40% and 50%, at least 45%, at least 50%, between 50% and 60%, at least about 60%, between 60% and 70%, between 70% and 80%, at least 75%, at least about 80%, between 80% and 90%, at least about 90%, between 90% and 100%, at least 100%, between 100% and 200%, at least 200%, at least about 300%, at least about 400% or more higher than the wild type or control.

As used herein, the term “before”, in reference to a sequence position, refers to an occurrence of one sequence upstream, or 5′, to another sequence.

Efficiency is a measure of enzyme activity relative to the theoretical limit of diffusion-limited substrate binding to the enzyme (Johnson et al. 2019). Herein the term “efficiency” is used to refer to the steady-state kinetic parameter, k_{cat}/Kₘ, which is the apparent second-order rate constant for substrate binding and conversion to product. Kinetic parameters derived using direct methods as described in Gong et al. 2018, Liu et al. 2020, and Bravo et al, 2022 (herein incorporated by reference in their entirety) are implicitly given.

By “specificity” is meant a function of the efficiency of reaction for a desired substrate relative to that for an undesired substrate (Johnson et al. 2019; Liu et al. 2020; Liu et al., 2019; Gong et al. 2018). Mathematically, efficiency is defined as the ratio of k_{cat}/Kₘ values to the two substrates.

By “variant” or “fragment” is meant a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, or at least 99% sequence identity to a parent Cas12a2 polypeptide. It is noted that “parent” and “native” are referred to alternatively herein, and have the same meaning, which is the naturally occurring Cas12a2 on which the variant or fragment thereof is based.

### General Description

Disclosed herein are variants of SEQ ID NO: 1 which can have 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% homology with SEQ ID NO: 1, or any amount above, below, or between these percentages. Put another way, contemplated herein are variants of SEQ ID NO: 1 with 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 or more amino acid residues that vary from SEQ ID NO: 1.

The isolated Cas12a2 molecule contemplated herein can have at least one mutation which provides the ability to cleave double stranded nucleic acid at a reduced rate compared to native Cas12a2. This means that the engineered, or mutated, Cas12a2 of the invention can retain the ability to cleave single stranded nucleic acid (either RNA or DNA, or a hybrid thereof) at or near the levels found in native Cas12a2, but that other forms of nucleic acid are cleaved at a reduced rate, or not at all. By “reduced rate” is meant that the rate of cleavage is reduced by 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% 40%, 4%1%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to the cleavage rate of native Cas12a2. The isolated Cas12a2 variant or fragment can have 1, 2, 3, 4, 5, 6, 7, 8, 9, 10, 11, 12, 13, 14, 15, 16, 17, 18, 19, or 20 or more amino acid variations when compared to the original, or native, Cas12a2 as represented by SEQ ID NO: 1. Examples of residues with this capability include, but are not limited to, Y465 and Y1080. Either of these residues can be mutated to residues which decrease the ability of the molecule to cleave double stranded nucleic acid at a reduced rate. One of skill in the art can appreciate what those mutations can entail. For example, Y465 can be mutated to Y465A. Y1080 can also be mutated. One example of such a mutation is Y1080A, but other residues could also be used and be equally or more effective. Mutations can exist in other sites as well as these sites.

The isolated Cas12a2 molecule contemplated herein can have at least one mutation which provides the ability to cleave double stranded nucleic acid as well as single stranded RNA at a reduced rate compared to native Cas12a2. This means that the engineered, or mutated, Cas12a2 of the invention can retain the ability to cleave single stranded DNA at or near the levels found in native Cas12a2, but that other forms of nucleic acid are cleaved at a reduced rate, or not at all. By “reduced rate” is meant that the rate of cleavage is reduced by 1%, 2%, 3%, 4%, 5%, 6%, 7%, 8%, 9%, 10%, 11%, 12%, %13%, %14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to the cleavage rate of native Cas12a2. It can be the case that the rates are different for double stranded nucleic acid and single stranded RNA. The isolated Cas12a2 variant or fragment can have 1, 2, 3, 4, 5, 6, 7, 8, 9, 10, 11, 12, 13, 14, 15, 16, 17, 18, 19, or 20 or more amino acid variations when compared to the original, or native, Cas12a2 as represented by SEQ ID NO: 1. An example of a residue with this capability includes, but is not limited to, Y1069. One of skill in the art can appreciate what those mutations can entail. For example, Y1069 can be mutated to Y1069A. Mutations can exist in other sites as well as these sites.

Also disclosed is a mutation at F1092, which can reduce all cleavage. An example is F1092A. By “reducing all cleavage” is meant that cleavage is reduced by %, 2%, 3%, 4%, 5%, 6%, 7%, 8%, 9%, 10%, 11%, 12%, 13%, 14%, 15%, %16%, %17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% compared to the cleavage rate of native Cas12a2. For example, it can be completely abolished.

Also disclosed herein is a method of cleaving a single stranded nucleic acid, the method comprising: a) providing a Cas12a2 or functional variant, wherein said Cas12a2 or functional variant thereof is capable of indiscriminately cleaving single stranded nucleic acid upon recognition of a specific complementary RNA target, and further wherein one or more residues are mutated such that double stranded nucleic acid is cleaved at a reduced rate compared to native Cas12a2; b) providing an RNA target; c) providing single stranded nucleic acid other than the RNA target; and d) exposing the isolated protein of step a) to the RNA target of step b) and the single stranded nucleic acid of step c), wherein the isolated protein cleaves the single stranded nucleic acid in the presence of the RNA target, and cleaves double stranded nucleic acid at a reduced rate compared to native Cas12a2.

The Cas12a2 or functional mutant thereof can be one of the molecules described herein. For example, the molecule can be 80%, 90%, 95%, or more identical to SEQ ID NO: 1. More specifically, residue Y465 and/or residue Y1080 can be mutated with respect to SEQ ID NO: 1. Even more specifically, the mutation can be Y465A and/or Y1080A. Other mutations which confer desired benefits are also contemplated. The specific complementary RNA target can be recognized by crRNA. For example, the crRNA can bind to the isolated protein, wherein this interaction allows cleavage of single stranded nucleic acid. The specific complementary RNA target can comprise a protospacer-flanking sequence (PFS). Such PFS molecules are known in the art.

Also disclosed herein is a method of cleaving a single stranded DNA, the method comprising: a) providing a Cas12a2 or functional variant, wherein said Cas12a2 or functional variant thereof is capable of indiscriminately cleaving single stranded DNA upon recognition of a specific complementary RNA target, and further wherein one or more residues are mutated such that double stranded nucleic acid and single stranded RNA are cleaved at a reduced rate compared to native Cas12a2; b) providing an RNA target; c) providing single stranded DNA; and d) exposing the isolated protein of step a) to the RNA target of step b) and the single stranded DNA of step c), wherein the isolated protein cleaves the single stranded DNA in the presence of the RNA target, and cleaves double stranded nucleic acid and single stranded RNA at a reduced rate compared to native Cas12a2.

Again, the Cas12a2 or functional mutant thereof can be one of the molecules described herein. For example, the molecule can be 80%, 90%, 95%, or more identical to SEQ ID NO: 1. More specifically, residue Y1069 can be mutated with respect to SEQ ID NO: 1. Even more specifically, the mutation can be Y1069A. As described above, the specific complementary RNA target can be recognized by crRNA. The crRNA can bind to the isolated protein, wherein this interaction allows cleavage of single stranded DNA. The specific complementary RNA target can comprise a PFS.

This method of cleaving can be used to detect nucleic acid. When this occurs, the single stranded nucleic acid sequence (for example) can be labeled, such that cleavage is detectable. This molecule is referred to herein as the “detector nucleic acid.” The method can be used to detect disease, such as the presence of a pathogen. More detail follows regarding methods of detecting nucleic acids.

In some cases, the detection method includes a step of measuring the effect of a cleavage event. A detectable signal can be any signal that is produced when single-stranded nucleic acid is cleaved. For example, in some cases the step of measuring can include one or more of: gold nanoparticle based detection (e.g., see Xu et al., Angew Chem Int Ed Engl. 2007; 46(19):3468-70; and Xia et al., Proc Natl Acad Sci USA. 2010 Jun. 15; 107(24):10837-41), fluorescence polarization, colloid phase transition/dispersion (e.g., Baksh et al., Nature. 2004 Jan. 8; 427(6970):139-41), electrochemical detection, semiconductor-based sensing (e.g., Rothberg et al., Nature. 2011 Jul. 20; 475(7356):348-52; e.g., one could use a phosphatase to generate a pH change after ssDNA cleavage reactions, by opening 2′-3′ cyclic phosphates, and by releasing inorganic phosphate into solution), and detection of a labeled detector ssDNA. The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

Once the detector nucleic acid is cleaved, the detection moiety can be released or separated from the reporter and generates a detectable signal. In one embodiment, the detection moiety can be immobilized on a support medium. As described above, the detection moiety can be at least one of a fluorophore, a dye, a polypeptide, or a nucleic acid. Sometimes the detection moiety binds to a capture molecule on the support medium to be immobilized. The detectable signal can be visualized on the support medium to assess the presence or level of the target nucleic acid associated with an ailment, such as a disease, cancer, or genetic disorder.

The measuring can in some cases be quantitative, e.g., in the sense that the amount of signal detected can be used to determine the amount of target RNA present in the sample. The measuring can in some cases be qualitative, e.g., in the sense that the presence or absence of detectable signal can indicate the presence or absence of targeted RNA (e.g., virus). In some cases, a detectable signal will not be present (e.g., above a given threshold level) unless the targeted RNA(s) is present above a particular threshold concentration. In some cases, the threshold of detection can be titrated by modifying the amount of engineered Cas12a12, guide RNA, sample volume, and/or detector nucleic acid (if one is used). As such, for example, as would be understood by one of ordinary skill in the art, a number of controls can be used if desired in order to set up one or more reactions, each set up to detect a different threshold level of target RNA, and thus such a series of reactions could be used to determine the amount of target RNA present in a sample (e.g., one could use such a series of reactions to determine that a target RNA is present in the sample ‘at a concentration of at least X’). The compositions and methods of this disclosure can be used to detect any RNA target.

In some cases, a method of the present disclosure can be used to determine the amount of a target RNA in a sample (e.g., a sample comprising the target RNA and a plurality of non-target RNAs). Determining the amount of a target RNA in a sample can comprise comparing the amount of detectable signal generated from a test sample to the amount of detectable signal generated from a reference sample. Determining the amount of a target RNA in a sample can comprise: measuring the detectable signal to generate a test measurement; measuring a detectable signal produced by a reference sample to generate a reference measurement; and comparing the test measurement to the reference measurement to determine an amount of target RNA present in the sample.

A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system.

Described herein are devices, systems, fluidic devices, kits, and methods for detecting the presence of a target nucleic acid in a sample. The devices, systems, fluidic devices, kits, and methods for detecting the presence of a target nucleic acid in a sample can be used in a rapid test (e.g., lab test or point-of-care test) for detection of a target nucleic acid of interest. For example, disclosed herein are particular microfluidic devices, lateral flow devices, sample preparation devices, and compositions (e.g., programmable nucleases, guide RNAs, reagents for in vitro transcription, reagents for amplification, reagents for reverse transcription, reporters, or any combination thereof) for use in said devices that are particularly well suited to carrying out a highly efficient, rapid, and accurate for detecting whether a target nucleic acid is present in a sample. In particular, provided herein are devices, systems, fluidic devices, and kits, wherein the rapid tests can be performed in a single system.

Specifically disclosed is a kit. This kit can comprise a Cas12a2 molecule, wherein said Cas12a2 molecule has been modified such that it indiscriminately cleaves single stranded nucleic acid upon recognition of a specific complementary RNA target, and further wherein one or more residues are mutated such that double stranded nucleic acid is cleaved at a reduced rate compared to native Cas12a2.

Also disclosed is a kit comprising a Cas12a2 molecule, wherein said Cas12a2 molecule has been modified such that it indiscriminately cleaves single stranded DNA upon recognition of a specific complementary RNA target, and further wherein one or more residues are mutated such that double stranded nucleic acid and single stranded RNA is cleaved at a reduced rate compared to native Cas12a2.

As described above, the Cas12a2 or functional mutant thereof can be one of the molecules described herein. For example, the molecule can be 80%, 90%, 95%, or more identical to SEQ ID NO: 1. More specifically, residue Y465 and/or residue Y1080 can be mutated with respect to SEQ ID NO: 1. Even more specifically, the mutation can be Y465A and/or Y1080A. Other mutations which confer desired benefits are also contemplated. The single-stranded nucleic acid can be RNA or DNA. The kit described herein can further comprise labeled single stranded nucleic acid for detection. It can further comprise crRNA comprising a sequence which recognizes target nucleic acid.

By “reduction in cleavage” is meant a reduction of overall cleavage which takes place can be 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, reduction, or any amount below or in-between these amounts. In one particular example, the cleavage rate can be reduced by 10%.

As mentioned above, the methods disclosed herein can be used to detect nucleic acid. Detecting nucleic acid can have a wide range of applications, which are known to those of skill in the art. One specific example is detecting genetic anomalies or somatic mutations. Detecting certain genetic anomalies or somatic mutations can lead to differential treatment, so that detection of a certain mutation can lead to a different method of treatment. An example is a cancer specific mutation, wherein the mutation may confer drug resistance.

Another application is in detecting disease. For example, the disease can be an infection, an organ disease, a blood disease, an immune system disease, a cancer, a brain and nervous system disease, an endocrine disease, a pregnancy or childbirth-related disease, an inherited disease, or an environmentally-acquired disease. In still further embodiments, the disease state is cancer or an autoimmune disease or an infection.

In further embodiments, the infection is caused by a virus, a bacterium, or a fungus, or the infection is a viral infection. In specific embodiments, the viral infection is caused by a double-stranded RNA virus, a positive sense RNA virus, a negative sense RNA virus, a retrovirus, or a combination thereof, or the viral infection is caused by a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae, or a Deltavirus, or the viral infection is caused by Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Borna disease virus, Ebola virus, Marburg virus, Measles virus, Mumps virus, Nipah virus, Hendra virus, Newcastle disease virus, Human respiratory syncytial virus, Rabies virus, Lassa virus, Hantavirus, Crimean-Congo hemorrhagic fever virus, Influenza, or Hepatitis D virus.

To further illustrate the principles of the present disclosure, the following examples are put forth so as to provide those of ordinary skill in the art with a complete disclosure and description of how the compositions, articles, and methods claimed herein are made and evaluated. They are intended to be purely exemplary of the invention and are not intended to limit the scope of what the inventors regard as their disclosure. Efforts have been made to ensure accuracy with respect to numbers (e.g., amounts, temperatures, etc.); however, some errors and deviations should be accounted for. Unless indicated otherwise, temperature is ° C. or is at ambient temperature, and pressure is at or near atmospheric.

There are numerous variations and combinations of process conditions that can be used to optimize product quality and performance. Only reasonable and routine experimentation will be required to optimize such process conditions.

### EXAMPLES

### Example 1: Large-scale Structural Rearrangements Activate Cas12a2 Indiscriminate Nuclease Activity

### Structure of Cas12a2 Binary Complex

To understand the unique mechanisms of activation and substrate accommodation by Cas12a2, biochemical and structural analyses were performed, including determining cryo-electron microscopy (cryo-EM) structures of autoinhibited Cas12a2-crRNA (binary complex), associated with an RNA target (ternary complex), and bound to both an RNA target and a double-stranded DNA collateral substrate mimetic.

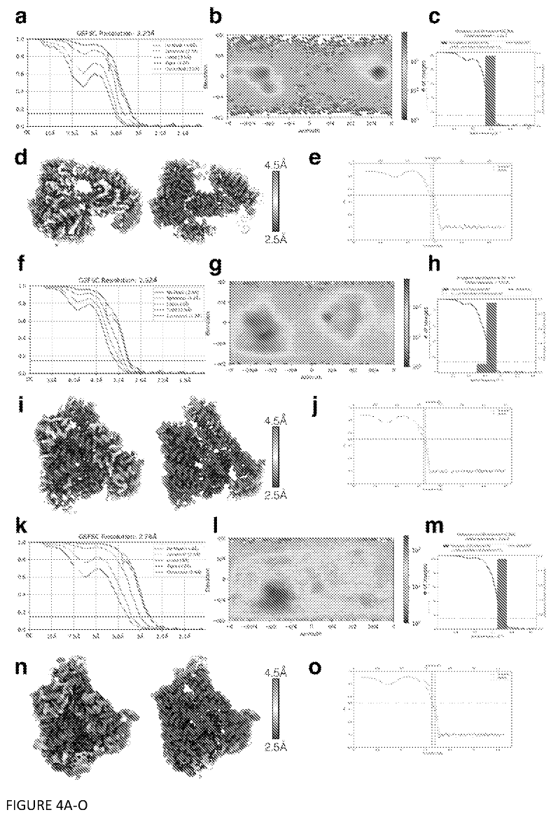

To gain structural insights into Cas12a2 function a binary complex was purified consisting of catalytically active Cas12a2 and a mature crRNA and determined the structure using cryo-electron microscopy (cryo-EM) to a global resolution of 3.2 Å (FIG. 4). The quality of the map allowed de novo modelling of the majority of Cas12a2, apart from the zinc ribbon (ZR) and protospacer-flanking sequence (PFS)-interacting (PI) domain. Both ZR and PI domains are important for substrate recognition and are flexible in the absence of target, as has been reported for Cas12a (Saha et al., 2020).

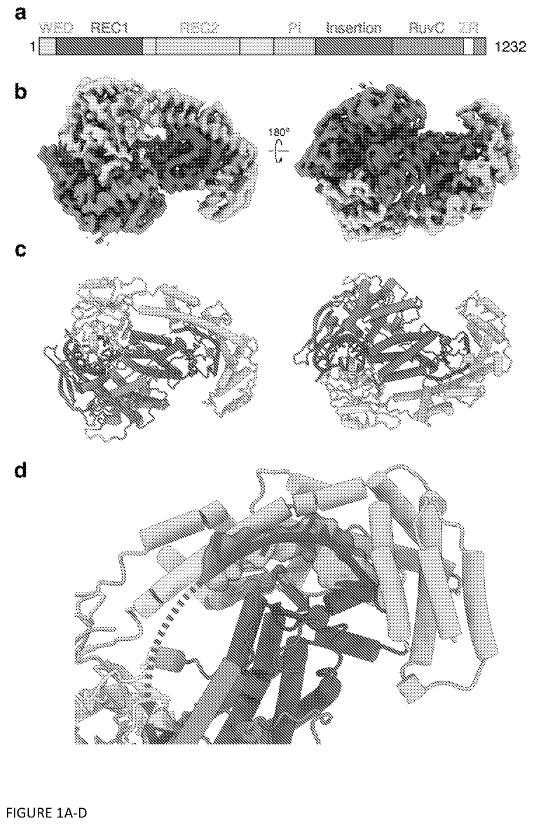

The overall structure of Cas12a2 can be divided into two lobes (FIG. 1), with a recognition (REC) lobe comprising the REC1 and REC2 domains and the nuclease (NUC) lobe consisting of the PI domain, wedge (WED), RuvC nuclease, ZR and the Cas12a2-specific insertion (“Insertion”). The overall architecture of the binary complex resembles an oyster, as opposed to the triangular “sea conch” shape of Cas12a (Stella et al., 2017).

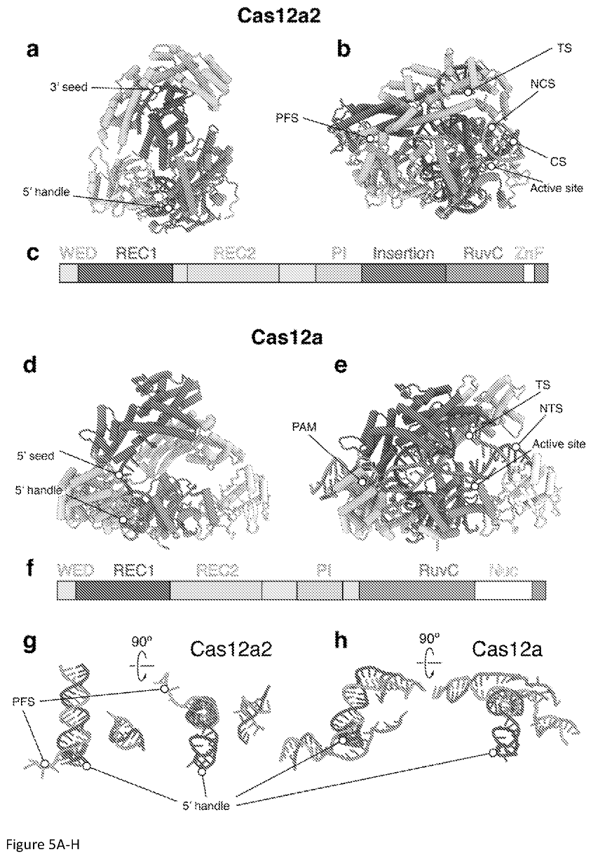

While Cas12a and Cas12a2 share low (10-20%) sequence similarity, comparison of the WED and Nuc domains shows a high degree of structural similarity (RMSD 1.073 Å across 120 equivalent residues with FnCas9 PDB 5NG6) (FIG. 5). Furthermore, the crRNA 5′ stem loop is in an identical configuration in both complexes. The common “chassis” formed by these domains provides a structural framework that enables the same crRNAs to prime and guide either complex to the same target sequences for their different functions.

Despite the similar domain organization to Cas12a within the NUC lobe, Cas12a2 has a unique α-helical REC lobe, with no known structural homologs. The differences in the structural organization of the REC lobe likely allow Cas12a2 to escape targeting by many anti-CRISPR (Acr) proteins that can efficiently shut down Cas12a. Interestingly, a 7-nt crRNA seed sits at the interface between REC1 and REC2 in a pre-ordered conformation where bases are solvent exposed and thus primed for target recognition (FIG. 1d). This seed is towards the 3′ end of the crRNA, and the intervening sequence between the 5′ crRNA stem-loop and 3′ seed is disordered and flexible in our structure. This is in stark contrast to Cas12a, which has a well-described 5′ crRNA seed region immediately flanking the 5′ stem-loop. In Cas12a, this region is highly sensitive to mismatches since it initiates R-loop formation upon PAM recognition (Strohkendl et al., 2018). On the contrary, Cas12a2 is insensitive to single mismatches within the 5′ end of the crRNA but has reduced in vivo activity in the presence of 3′ mismatches. This structure shows that Cas12a2 crRNA: target strand (TS) duplex formation can initiate and propagate from the 3′ end of the crRNA.

### RNA Target Binding Activates Cas12a2

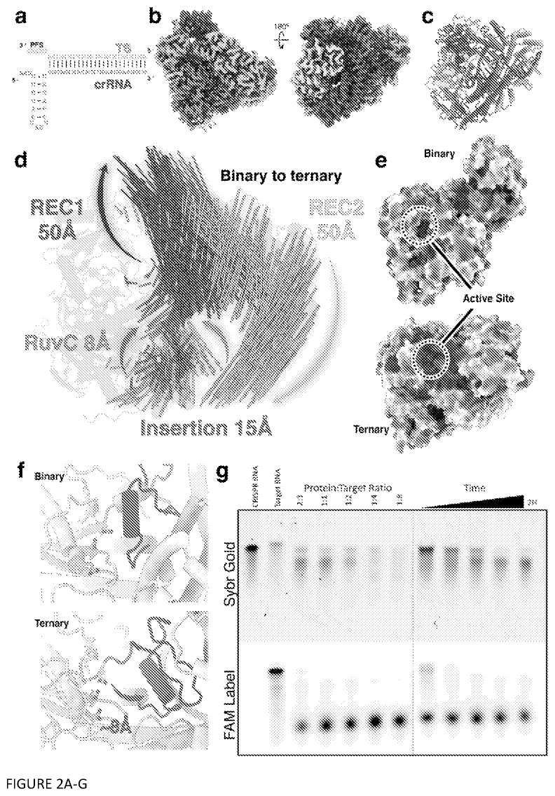

The binary complex structure indicated that RNA target recognition by Cas12a2 is distinct from other Cas12 family members. To understand how target binding activates Cas12a2, a 2.9 Å-resolution cryo-EM structure of a ternary complex containing Cas12a2, crRNA and a complementary ssRNA target strand (TS) containing a non-self protospacer-flanking sequence (PFS, 5′-GAAAG-3′) (FIG. 2) was determined. This TS activates the nuclease activity of Cas12a2. This structure exhibits well-ordered density for the PI domain, while the ZR domain is flexible as in the binary complex.

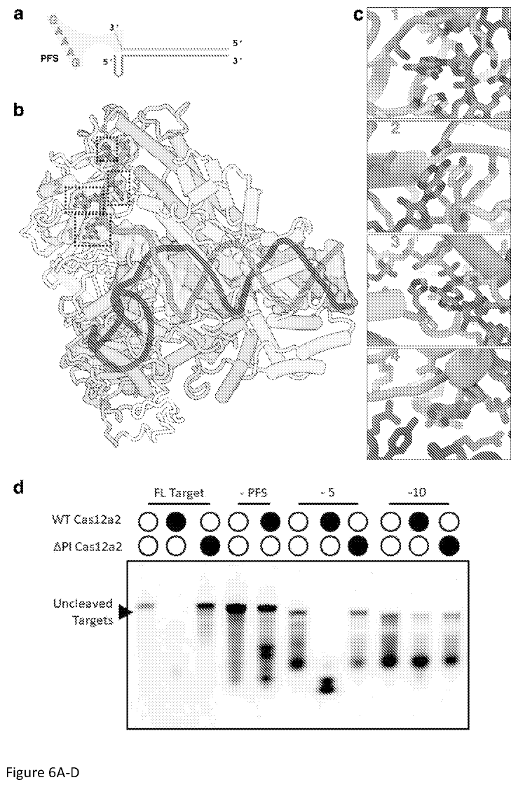

The 22-bp A-form crRNA:TS duplex runs through the center of the complex. At the 5′ end of the spacer, the duplex splits with the crRNA 5′ stem loop wedged between RuvC and WED domains, while the 3′ PFS end of the TS is gripped by the PI domain. Each of the 5 nts of the PFS make several specific base contacts with residues within the PI domain, including hydrogen bonding and π-π stacking (FIG. 6). These contacts stabilize the otherwise flexible PI domain, allowing Cas12a2 to distinguish self- (i.e., complementary to the crRNA 5′ handle) from non-self TS based on the PFS. This is further supported by the observation that removal of the PI domain did not affect the overall structure of Cas12a2 but abrogated nuclease activity (FIG. 6).

Cas12a2 is unable to degrade nucleic acids in the absence of a suitable TS RNA. Superposition of the binary and ternary complexes reveals substantial conformational changes localized to the REC1 and REC2 domains, while the NUC lobe remains predominantly static (FIG. 2d). REC1 and REC2 are both displaced by up to ˜50 Å, but move in different directions, creating a central channel which accommodates the crRNA:TS duplex. The Insertion domain moves by up to ˜15 Å, but these changes are exclusively localized to the C-terminal half of the domain (residues 938-1030). The N-terminal half (residues 870-937), which makes numerous contacts with the crRNA 5′ stem-loop, remains static. Based on this observation, it appears that the insertion domain acts as a transducer, allowing allosteric communication between the REC and NUC lobes in response to TS binding.

Inspection of the RuvC active site in the inactive binary complex reveals that the catalytic triad (D848, E1063, D1213) are buried within a solvent-excluded pocket. Strikingly, the conformational changes that accompany TS binding create a 25 Å-wide positively-charged groove that exposes the active site. This groove is of sufficient size to accommodate both ss- and ds-nucleic acids. While other Cas12 proteins undergo conformational changes upon crRNA hybridization (the largest is ˜25 Å (Pausch et al., 2021)), the changes observed in Cas12a2 are considerably larger.

Access to the RuvC catalytic triad is also mediated by the ˜8 Å shift of a lid helix, which contributes to the change in active site solvent exposure. This is akin to the lid loop or helix that gates active site exposure reported for other Cas12 endonucleases (Stella et al., 2018; Zhang et al., 2020).

Once activated by target binding, the lid of these endonucleases remains ‘open’, enabling ssDNA cleavage in trans. However, in previously reported Cas12a structures, the RuvC active site is still somewhat buried due to the presence of the Nuc domain (Swarts and Jinek, 2019). In a structure of catalytically dead Cas12i, trans ssDNA is tightly interwoven to sit within the active site (Zhang et al., 2020). This is in stark contrast to the highly accessible Cas12a2 RuvC active site in the ternary complex, providing a structural basis for efficient cleavage of a wide range of substrates in trans. The lack of Nuc domain and highly exposed RuvC active site in the ternary structure explain why Cas12a2 collateral nuclease activation results in a cell death abortive infection phenotype (cite co-submitted paper?), while Cas12a collateral ssDNase activity does not play a role in bacterial immunity (Marino et al., 2022).

The path followed by the TS completely circumvents the RuvC active site, suggesting that RNA degradation is predominantly in trans. To test this, the Cas12a2 binary complex was incubated with fluorescent TS RNA at a range of molar ratios and analyzed cleavage. The 5′ FAM label was consistently trimmed due to the ˜20-nt flexible RNA sequence extending from the spacer. Sybr-gold staining of the gel revealed that even though the FAM label was removed, the majority of the TS was largely protected from degradation when it was not in molar excess of Cas12a2 (i.e., when Cas12a2 was in excess or at molar equivalence). However, when the TS was in stoichiometric excess of Cas12a2, full degradation was observed (FIG. 2g). Furthermore, at a 2:1 Cas12a2 binary complex to TS ratio, the trimmed but intact TS persisted after 2 hours (a timepoint sufficient for complete plasmid DNA degradation). This indicates that Cas12a2 is activated for cleavage by the TS, but the majority of Cas12a2 cleavage is in trans. This is distinct from the cleavage mechanisms of other Cas12 nucleases, and is reminiscent of Cas13 RNAse activity in cis and trans.

Collateral dsDNA Binding Via Duplex Contortion

While the binary and ternary complexes reveal intricate mechanisms of TS-alleviated autoinhibition of Cas12a2, until now, it remained unclear how Cas12a2 degrades duplexed nucleic acids. While the active site cleft is large enough to accommodate duplexes, the scissile phosphate must be carefully positioned within the RuvC nuclease domain to enable catalysis.

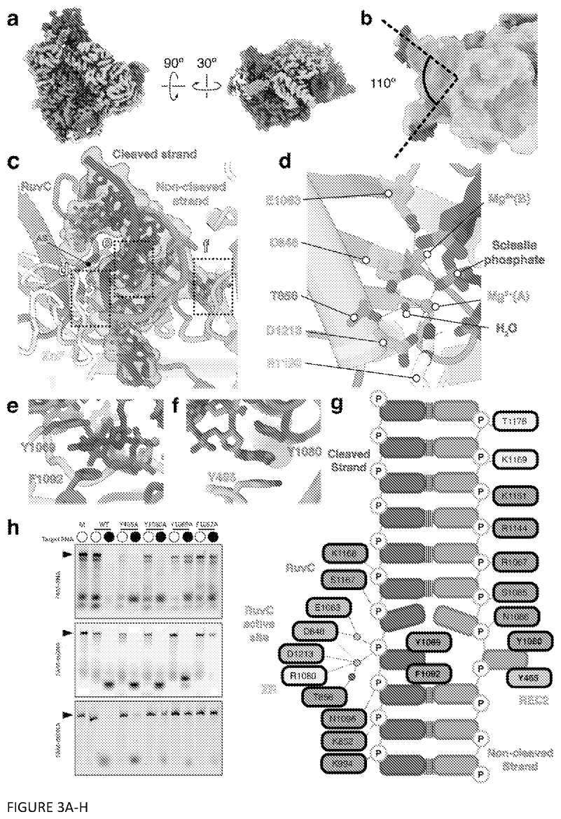

To directly visualize how Cas12a2 accommodates double-stranded DNA substrates, a 2.8 Å resolution structure of crRNA-guided Cas12a2 bound to both an activating TS and a collateral dsDNA substrate (FIG. 3) was determined. The RuvC active site and 11 of 20 bp of a DNA duplex were well resolved, while the flexible DNA ends are only visible at lower density thresholds. Due to averaging of different duplex registers, it was not possible to ascribe sequence positions, and the duplex was modeled as polyA:T.

Unexpectedly, it was observed that the RuvC and Rec domains bind the dsDNA duplex in a bent conformation that kinks the duplex 110°, and forces the phopshodiester backbone of the duplex into the RuvC active site (FIG. 3b). The duplex is distorted through the local melting of two base pairs in the immediate vicinity of the active site. The DNA strand within the RuvC active site was designated as the cleaved strand (CS), and the complementary DNA strand as the non-cleaved strand (NCS) to differentiate from TS and NTS nomenclature used to describe the strands of dsDNA that are specifically targeted with a crRNA guide (e.g. Cas12a or Cas9).

Both the CS and NCS are stabilized by a network of non-specific interactions with Cas12a2 (FIG. 3). This myriad of contacts with both duplex ends likely results in duplex bending, and local melting. The melted bases are subsequently captured by two pairs of ‘aromatic clamps’ (Y465 and Y1080, Y1069 and F1092) that each hold a single DNA base through π-π stacking, preventing re-hybridization.

The importance of the aromatic clamps were tested through site-directed mutagenesis and cleavage assays, where each of the four clamp residues were individually substituted for alanine. Wild-type Cas12a2 can cleave collateral ssRNA, ssDNA, and dsDNA upon incubation of activating TS RNA (FIG. 3h). Mutation of the NCS clamp residue Y465 did not affect the cleavage of ssRNA and ssDNA, but reduced the ability of Cas12a2 to cleave dsDNA. This effect was more pronounced with the NCS clamp Y1080A mutation, which strongly reduced dsDNA cleavage yet did not affect ssRNA and DNA cleavage. Mutation of either CS clamp residue (Y1069A or F1092A) blocked cleavage of both dsDNA and ssRNA, indicating that this clamp is pivotal for both substrate unwinding and positioning the CS within the active site. Interestingly, F1092A abolished all cleavage, Y1069A was unique in that it abolished dsDNA and ssRNA cleavage but did not abolish ssDNA cleavage. This shows that F1092 is analogous to the single aromatic residue often found in RuvC sites that positions the target nucleotide for cleavage (Bravo et al., 2022; Górecka et al., 2019; Huang et al., 2020), but the additional aromatic residue Y1069 plays a role in stabilizing the distorted duplex conformation to facilitate nuclease activity.

Since both DNA strands contained non-hydrolysable phosphothioate modifications, visualize the pre-hydrolysis RuvC active site state was visualized, including two Mg²⁺ ions and a putative activating water adjacent to Mg²⁺ (A) (FIG. 3). Interestingly, R1180 from the ZR domain is involved in coordinating Mg²⁺ (A), and is in the ‘down’ position as seen for an equivalent arginine in the Cas9 RuvC site (Bravo et al., 2022; Casalino et al., 2020). The role of the ZR in collateral cleavage is supported by the increased quality of the ZR density in the quaternary complex compared to the binary or ternary complexes, and the observation that mutation of any of the Zn²⁺-coordinating Cys residues abrogates Cas12a2 nuclease activity.

The catalytic mechanism of Cas12a2 is consistent with that of other RuvC endonucleases (Bravo et al., 2022; Górecka et al., 2019; Huang et al., 2020; Pausch et al., 2021), where protein-induced structural tension of the DNA facilitates proper scissile phosphate coordination. However, within the available Cas9 and Cas12a2 structures, the NCS aromatic clamps provide a unique strategy to cleave duplexed nucleic acids.

### Discussion

Provided herein is a molecular mechanism for the unique activity of Cas12a2. These include:
1. Differences in crRNA seed and PI domain can prevent R-loop formation and activation by dsDNA.
2. TS binding is accompanied by large scale conformational changes that enable activation. Lack of Nuc domain increases exposure of active site, enabling dsDNase activity. While target recognition by Cas12a can activate single-stranded DNase activity in trans (Chen et al., 2018; Swarts and Jinek, 2019), this cleavage is slow and likely does not play a role in bacterial immunity (Marino et al., 2022).
3. Since Cas12a and Cas12a2 co-occur, Cas12a2 can function as a sophisticated backup-system in instances of immune evasion. If stringent DNA surveillance by Cas12a is blocked through Acrs or mutagenic escape and the phage can transcribe RNA, Cas12a2 can utilize the same crRNA and target phage RNA while evading AcrVA action and tolerating mismatches. Cas12a2 activation then triggers abortive infection, preventing phage replication and assembly. This achieves population-level defense.
4. The discovery of the Y1069Aa point mutant and how it provides a needed RNA-sensor with collateral activity that doesn't cleave the RNA-target can developed as an RNA-diagnostic.
5. The PFS and PFS interacting domain and how self vs. non-self recognition seems to be distinct from other RNA sensing systems that use base-pairing with self to deactivate while, lack of a PFS is often activating.
6. RuvC domains all have to distort DNA to achieve this. No similar distortion of duplex is seen, because NTS is completely unwound (i.e. is ssDNA) when accommodated into Cas12a/b/e/f/g/h/i/j active site. Same with Cas9 RuvC and NTS.
7. No secondary messengers—Cas12a2 is sufficient to trigger Abi.
8. Immunity to Acrs.

Lastly, it should be understood that while the present disclosure has been provided in detail with respect to certain illustrative and specific aspects thereof, it should not be considered limited to such, as numerous modifications are possible without departing from the broad spirit and scope of the present disclosure as defined in the appended claims.

### Tables

| | Binary | Ternary | Quaternary |
|---|---|---|---|
| | complex | complex | complex |
| Data collection | | | |
| and processing | | | |
| Voltage (kV) | 200 | 300 | 200 |
| Electron exposure | 40 | 80 | 40 |
| (e−/Å²) | | | |
| Defocus range | −1.5 to −2.5 | | |
| (μm) | | | |
| Pixel size (Å) | 0.94 | 0.81 | 0.94 |
| Symmetry imposed | | | |
| Initial particle | 981,772 | 2,996,634 | 1,692,836 |
| images (no.) | | | |
| Final particle | 80,528 | 192,639 | 94,805 |
| images (no.) | | | |
| Map resolution (Å) | 3.20 | 3.47 | 2.78 |
| FSC threshold | | | |
| Map resolution range (Å) | | | |
| Refinement | | | |
| Initial model used | N/A | N/A | N/A |
| (PDB code) | | | |
| Model resolution | 3.3 | 3.0 | 3.0 |
| (Å) | | | |
| FSC threshold | 0.5 | 0.5 | 0.5 |
| Map sharpening B | 102.6 | 90.3 | 85.4 |
| factor (Å²) | | | |
| Model composition | 9305 | 11309 | 11739 |
| Non-hydrogen | 1075 | 1203 | 1203 |
| atoms | | | |
| Protein residues | 26 | 69 | 90 |
| Nucleotides | | | |
| Ligands | | | |
| Mean B factors (Å²) | | | |
| Protein | 71.98 | 18.50 | 34.76 |
| Nucleotides | 56.64 | 16.73 | 41.51 |
| R.m.s. deviations | | | |
| Bond lengths | 0.007 | 0.006 | 0.006 |
| (Å) | | | |
| Bond angles (°) | 0.661 | 0.596 | 0.588 |
| Validation | | | |
| MolProbity | 1.49 | 1.51 | 1.41 |
| score | | | |
| Clashscore | 5.46 | 6.92 | 4.18 |
| Poor rotamers | 0 | 0 | 0 |
| (%) | | | |
| Ramachandran plot | | | |
| Favored (%) | 96.77 | 97.33 | 96.66 |
| Allowed (%) | 3.23 | 2.67 | 3.34 |
| Disallowed (%) | 0 | 0 | 0 |
| Map CC (mask) | 0.88 | 0.81 | 0.83 |

### REFERENCES

Bravo, J. P. K., Liu, M.-S., McCool, R. S., Jung, K., Johnson, K. A., and Taylor, D. W. (2022). Structural basis for mismatch surveillance by CRISPR/Cas9. Nature.
Casalino, L., Nierzwicki, L., Jinek, M., and Palermo, G. (2020). Catalytic Mechanism of Non-Target DNA Cleavage in CRISPR-Cas9 Revealed by Ab Initio Molecular Dynamics. ACS Catal. 10, 13596-13605.
Chen, J. S., Ma, E., Harrington, L. B., Da Costa, M., Tian, X., Palefsky, J. M., and Doudna, J. A. (2018). CRISPR-Cas12a target binding unleashes indiscriminate single-stranded DNase activity. Science (80-.). 360, 436-439.
Górecka, K. M., Krepl, M., Szlachcic, A., Poznański, J., Šponer, J., and Nowotny, M. (2019). RuvC uses dynamic probing of the Holliday junction to achieve sequence specificity and efficient resolution. Nat. Commun. 10.
Hampton, H. G., Watson, B. N. J., and Fineran, P. C. (2020). The arms race between bacteria and their phage foes. Nature 577, 327-336.
Huang, X., Sun, W., Cheng, Z., Chen, M., Li, X., Wang, J., Sheng, G., Gong, W., and Wang, Y. (2020). Structural basis for two metal-ion catalysis of DNA cleavage by Cas12i2. Nat. Commun. 11.
Makarova, K. S., Wolf, Y. I., Iranzo, J., Shmakov, S. A., Alkhnbashi, O. S., Brouns, S. J. J., Charpentier, E., Cheng, D., Haft, D. H., Horvath, P., et al. (2020). Evolutionary classification of CRISPR-Cas systems: a burst of class 2 and derived variants. Nat. Rev. Microbiol. 18, 67-83.
Marino, N. D., Pinilla-redondo, R., and Bondy-denomy, J. (2022). CRISPR-Cas12a targeting of ssDNA plays no detectable role in immunity.
Pausch, P., Soczek, K. M., Herbst, D. A., Tsuchida, C. A., Al-Shayeb, B., Banfield, J. F., Nogales, E., and Doudna, J. A. (2021). DNA interference states of the hypercompact CRISPR-CasΦ effector. Nat. Struct. Mol. Biol. 28, 652-661.
Saha, A., Arantes, P. R., Hsu, R. V., Narkhede, Y. B., Jinek, M., and Palermo, G. (2020). Molecular Dynamics Reveals a DNA-Induced Dynamic Switch Triggering Activation of CRISPR-Cas12a. J. Chem. Inf. Model. 60, 6427-6437.
Stella, S., Alcón, P., and Montoya, G. (2017). Structure of the Cpf1 endonuclease R-loop complex after target DNA cleavage. Nature 546, 559-563.
Stella, S., Mesa, P., Thomsen, J., Paul, B., Alcón, P., Jensen, S. B., Saligram, B., Moses, M. E., Hatzakis, N. S., and Montoya, G. (2018). Conformational Activation Promotes CRISPR-Cas12a Catalysis and Resetting of the Endonuclease Activity. Cell 175, 1856-1871.e21.
Strohkendl, I., Saifuddin, F. A., Rybarski, J. R., Finkelstein, I. J., and Russell, R. (2018). Kinetic Basis for DNA Target Specificity of CRISPR-Cas12a. Mol. Cell 71, 816-824.e3.
Swarts, D. C., and Jinek, M. (2019). Mechanistic Insights into the cis- and trans-Acting DNase Activities of Cas12a. Mol. Cell 73, 589-600.e4.
Yan, W. X., Hunnewell, P., Alfonse, L. E., Carte, J. M., Keston-Smith, E., Sothiselvam, S., Garrity, A. J., Chong, S., Makarova, K. S., Koonin, E. V., et al. (2019). Functionally diverse type V CRISPR-Cas systems. Science (80-.). 363, 88-91.
Zhang, H., Li, Z., Xiao, R., and Chang, L. (2020). Mechanisms for target recognition and cleavage by the Cas12i RNA-guided endonuclease. Nat. Struct. Mol. Biol. 27, 1069-1076.

SEQUENCES

SEQ ID NO: 1: Full length Cas12a2:
MLHAFTNQYQLSKTLRFGATLKEDEKKCKSHEELKGFVDISYENMKSSATIAESLN
ENELVKKCERCYSEIVKFHNAWEKIYYRTDQIAVYKDFYRQLSRKARFDAGKQNS
QLITLASLCGMYQGAKLSRYITNYWKDNITRQKSFLKDFSQQLHQYTRALEKSDKA
HTKPNLINFNKTFMVLANLVNEIVIPLSNGAISFPNISKLEDGEESHLIEFALNDYSQL
SELIGELKDAIATNGGYTPFAKVTLNHYTAEQKPHVFKNDIDAKIRELKLIGLVETL
KGKSSEQIEEYFSNLDKFSTYNDRNQSVIVRTQCFKYKPIPFLVKHQLAKYISEPNG
WDEDAVAKVLDAVGAIRSPAHDYANNQEGFDLNHYPIKVAFDYAWEQLANSLYT
TVTFPQEMCEKYLNSIYGCEVSKEPVFKFYADLLYIRKNLAVLEHKNNLPSNQEEFI
CKINNTFENIVLPYKISQFETYKKDILAWINDGHDHKKYTDAKQQLGFIRGGLKGRI
KAEEVSQKDKYGKIKSYYENPYTKLTNEFKQISSTYGKTFAELRDKFKEKNEITKIT
HFGIIIEDKNRDRYLLASELKHEQINHVSTILNKLDKSSEFITYQVKSLTSKTLIKLIKN
HTTKKGAISPYADFHTSKTGFNKNEIEKNWDNYKREQVLVEYVKDCLTDSTMAKN
QNWAEFGWNFEKCNSYEDIEHEIDQKSYLLQSDTISKQSIASLVEGGCLLLPIINQDI
TSKERKDKNQFSKDWNHIFEGSKEFRLHPEFAVSYRTPIEGYPVQKRYGRLQFVCA
FNAHIVPQNGEFINLKKQIENFNDEDVQKRNVTEFNKKVNHALSDKEYVVIGIDRG
LKQLATLCVLDKRGKILGDFEIYKKEFVRAEKRSESHWEHTQAETRHILDLSNLRV
ETTIEGKKVLVDQSLTLVKKNRDTPDEEATEENKQKIKLKQLSYIRKLQHKMQTNE
QDVLDLINNEPSDEEFKKRIEGLISSFGEGQKYADLPINTMREMISDLQGVIARGNN
QTEKNKIIELDAADNLKQGIVANMIGIVNYIFAKYSYKAYISLEDLSRAYGGAKSGY
DGRYLPSTSQDEDVDFKEQQNQMLAGLGTYQFFEMQLLKKLQKIQSDNTVLRFVP
AFRSADNYRNILRLEETKYKSKPFGVVHFIDPKFTSKKCPVCSKTNVYRDKDDILVC
KECGFRSDSQLKERENNIHYIHNGDDNGAYHIALKSVENLIQMK

What is claimed is:

1. An isolated protein comprising Cas12a2 or a functional variant thereof, wherein said isolated protein is capable of indiscriminately cleaving single stranded nucleic acid upon recognition of a specific complementary RNA target, and further wherein one or more residues are mutated such that double stranded nucleic acid cleavage is reduced or abrogated compared to native Cas12a2.

2. The isolated protein of claim 1, wherein Cas12a2 is represented by a protein with 80% or more identity to SEQ ID NO: 1.

3. The isolated protein of claim 1 or 2, wherein residue Y465 is mutated.

4. The isolated protein of claim 3, wherein said mutation comprises a Y465A substitution.

5. The isolated protein of any of claims 1-4, wherein residue Y1080 is mutated.

6. The isolated protein of claim 5, wherein said mutation comprises a Y1080A substitution.

7. The isolated protein of any one of claims 2-6, wherein said protein is 90% or more identical to SEQ ID NO: 1.

8. The isolated protein of any one of claims 2-7, wherein said protein is 95% or more identical to SEQ ID NO: 1.

9. The isolated protein of any one of claims 1-8, wherein said single stranded nucleic acid is RNA or DNA.

10. The isolated protein of any one of claims 1-9, wherein said reduced rate comprises a 10% or more reduction in cleavage.

11. An isolated protein comprising Cas12a2 or a functional variant thereof, wherein said isolated protein is capable of indiscriminately cleaving single stranded DNA upon recognition of a specific complementary RNA target, and further wherein one or more residues are mutated such that double stranded nucleic acid and single stranded RNA are cleaved at a reduced rate compared to native Cas12a2.

12. The isolated protein of claim 11, wherein Cas12a2 is represented by a protein with 80% or more identity to SEQ ID NO: 1.

13. The isolated protein of claim 11 or 12, wherein residue Y1069 is mutated.

14. The isolated protein of claim 13, wherein said mutation comprises a Y1069A substitution.

15. The isolated protein of any one of claims 12-14, wherein said protein is 90% or more identical to SEQ ID NO: 1.

16. The isolated protein of any one of claims 12-15, wherein said protein is 95% or more identical to SEQ ID NO: 1.

17. The isolated protein of any one of claims 12-16, wherein said reduced rate comprises a 10% or more reduction in cleavage.

18. A method of cleaving a single stranded nucleic acid, the method comprising:
a. providing a Cas12a2 or functional variant, wherein said Cas12a2 or functional variant thereof is capable of indiscriminately cleaving single stranded nucleic acid upon recognition of a specific complementary RNA target, and further wherein one or more residues are mutated such that double stranded nucleic acid is cleaved at a reduced rate compared to native Cas12a2;
b. providing an RNA target;
c. providing single stranded nucleic acid other than the RNA target; and
d. exposing the isolated protein of step a) to the RNA target of step b) and the single stranded nucleic acid of step c), wherein the isolated protein cleaves the single stranded nucleic acid in the presence of the RNA target, and cleaves double stranded nucleic acid at a reduced rate compared to native Cas12a2.

19. The method of claim 18, wherein the Cas12a2 or functional variant thereof is represented by an isolated protein which is 80% or more identical to SEQ ID NO: 1.

20. The method of claim 18 or 19, wherein the single stranded nucleic acid is RNA or DNA.

21. The method of any one of claims 18-20, wherein the specific complementary RNA target is recognized by crRNA.

22. The method of claim 21, wherein the crRNA binds to the isolated protein, wherein this interaction allows cleavage of single stranded nucleic acid.

23. The method of any one of claims 18-22, wherein said specific complementary RNA target comprises a protospacer-flanking sequence.

24. The method of any one of claims 18-23, wherein residue Y465 is mutated.

25. The method of claim 24, wherein said mutation comprises a Y465A substitution.

26. The method of any one of claims 18-25, wherein residue Y1080 is mutated.

27. The method of claim 26, wherein said mutation comprises a Y1080A substitution.

28. The method of any one of claims 19-27, wherein said protein is 90% or more identical to SEQ ID NO: 1.

29. The method of any one of claims 19-28, wherein said protein is 95% or more identical to SEQ ID NO: 1.

30. The method of any one of claims 18-29, wherein said reduced rate comprises a 10% or more reduction in cleavage.

31. A method of cleaving a single stranded DNA, the method comprising:
a. providing a Cas12a2 or functional variant, wherein said Cas12a2 or functional variant thereof is capable of indiscriminately cleaving single stranded DNA upon recognition of a specific complementary RNA target, and further wherein one or more residues are mutated such that double stranded nucleic acid and single stranded RNA are cleaved at a reduced rate compared to native Cas12a2;
b. providing an RNA target;
c. providing single stranded DNA; and
d. exposing the isolated protein of step a) to the RNA target of step b) and the single stranded DNA of step c), wherein the isolated protein cleaves the single stranded DNA in the presence of the RNA target, and cleaves double stranded nucleic acid and single stranded RNA at a reduced rate compared to native Cas12a2.

32. The method of claim 31, wherein the Cas12a2 or functional variant thereof is represented by an isolated protein which is 80% or more identical to SEQ ID NO: 1.

33. The method of claim 31 or 32, wherein the specific complementary RNA target is recognized by crRNA.

34. The method of claim 33, wherein the crRNA binds to the isolated protein, wherein this interaction allows cleavage of single stranded DNA.

35. The method of any one of claims 31-34, wherein said specific complementary RNA target comprises a protospacer-flanking sequence.

36. The method of any one of claims 31-35, wherein residue Y1069 is mutated.

37. The method of claim 36, wherein said mutation comprises a Y1069A substitution.

38. The method of any one of claims 32-36, wherein said protein is 90% or more identical to SEQ ID NO: 1.

39. The method of any one of claims 32-37, wherein said protein is 95% or more identical to SEQ ID NO: 1.

40. The method of any one of claims 31-39, wherein said reduced rate comprises a 10% or more reduction in cleavage.

41. A method of detecting a target RNA sequence, the method comprising:
a. providing a Cas12a2 or functional variant, wherein said Cas12a2 or functional variant thereof is capable of indiscriminately cleaving single stranded nucleic acid upon recognition of a specific complementary RNA target, and further wherein one or more residues are mutated such that double stranded nucleic acid is cleaved at a reduced rate compared to native Cas12a2;
b. providing a sample which may contain the RNA target sequence;
c. providing single stranded nucleic acid other than the RNA target sequence;
d. exposing the isolated protein of step a) to the RNA target of step b) and the single stranded nucleic acid of step c), wherein the isolated protein cleaves the single stranded nucleic acid in the presence of the RNA target, and cleaves double stranded nucleic acid at a reduced rate compared to native Cas12a2; and
e. detecting cleavage of single stranded nucleic acid other than the RNA target, thereby detecting the presence of the target RNA sequence.

42. The method of claim 41, wherein the single stranded nucleic acid sequence is labeled, such that cleavage is detectable.

43. The method of claim 41 or 42, wherein the Cas12a2 or functional variant thereof is represented by an isolated protein which is 80% or more identical to SEQ ID NO: 1.

44. The method any one of claims 41-43, wherein the single stranded nucleic acid is RNA or DNA.

45. The method of any one of claims 41-44, wherein the specific complementary RNA target is recognized by crRNA.

46. The method of claim 45, wherein the crRNA binds to the isolated protein, wherein this interaction allows cleavage of single stranded nucleic acid.

47. The method of any one of claims 41-46, wherein said specific complementary RNA target comprises a protospacer-flanking sequence.

48. The method of any one of claims 41-47, wherein residue Y465 is mutated.

49. The method of claim 48, wherein said mutation comprises a Y465A substitution.

50. The method of any one of claims 41-49, wherein residue Y1080 is mutated.

51. The method of claim 50, wherein said mutation comprises a Y1080A substitution.

52. The method of any one of claims 43-51, wherein said protein is 90% or more identical to SEQ ID NO: 1.

53. The method of any one of claims 43-52, wherein said protein is 95% or more identical to SEQ ID NO: 1.

54. The method of any one of claims 41-53, wherein said reduced rate comprises a 10% or more reduction in cleavage.

55. The method of any one of claims 41-54, wherein said detection is used to detect disease.

56. The method of any one of claims 41-54, wherein said detection is used to detect the presence of a pathogen.

57. A method of detecting a target RNA sequence, the method comprising:
a. providing a Cas12a2 or functional variant, wherein said Cas12a2 or functional variant thereof is capable of indiscriminately cleaving single stranded DNA upon recognition of a specific complementary RNA target, and further wherein one or more residues are mutated such that double stranded nucleic acid and single strand RNA are cleaved at a reduced rate compared to native Cas12a2;
b. providing a sample which may contain the RNA target sequence;
c. providing single stranded nucleic acid other than the RNA target sequence;
d. exposing the isolated protein of step a) to the RNA target of step b) and the single stranded DNA of step c), wherein the isolated protein cleaves the single stranded DNA in the presence of the RNA target, and cleaves double stranded nucleic acid and single stranded RNA at a reduced rate compared to native Cas12a2; and
e. detecting cleavage of single stranded DNA, thereby detecting the presence of the target RNA sequence.

58. The method of claim 57, wherein the single stranded nucleic acid sequence is labeled, such that cleavage is detectable.

59. The method of claim 57 or 58, wherein the Cas12a2 or functional variant thereof is represented by an isolated protein which is 80% or more identical to SEQ ID NO: 1.

60. The method of any one of claims 57-59, wherein the specific complementary RNA target is recognized by crRNA.

61. The method of claim 60, wherein the crRNA binds to the isolated protein, wherein this interaction allows cleavage of single stranded DNA.

62. The method of any one of claims 57-61, wherein said specific complementary RNA target comprises a protospacer-flanking sequence.

63. The method of any one of claims 57-62, wherein residue Y1069 is mutated.

64. The method of claim 63, wherein said mutation comprises a Y1069A substitution.

65. The method of any one of claims 59-64, wherein said protein is 90% or more identical to SEQ ID NO: 1.

66. The method of any one of claims 59-65, wherein said protein is 95% or more identical to SEQ ID NO: 1.

67. The method of any one of claims 57-66, wherein said reduced rate comprises a 10% or more reduction in cleavage.

68. The method of any one of claims 57-67, wherein said detection is used to detect disease.

69. The method of any one of claims 57-67, wherein said detection is used to detect the presence of a pathogen.

70. A kit comprising a Cas12a2 molecule, wherein said Cas12a2 molecule has been modified such that it indiscriminately cleaves single stranded nucleic acid upon recognition of a specific complementary RNA target, and further wherein one or more residues are mutated such that double stranded nucleic acid is cleaved at a reduced rate compared to native Cas12a2.

71. The kit of claim 70, wherein Cas12a2 is represented by a protein with 80% or more identity to SEQ ID NO: 1.

72. The kit of claim 70 or 71, wherein residue Y465 is mutated.

73. The kit of claim 72, wherein said mutation comprises a Y465A substitution.

74. The kit of any one of claims 70-73, wherein residue Y1080 is mutated.

75. The kit of claim 74, wherein said mutation comprises a Y1080A substitution.

76. The kit of any one of claims 71-75, wherein said protein is 90% or more identical to SEQ ID NO: 1.

77. The kit of any one of claims 71-76, wherein said protein is 95% or more identical to SEQ ID NO: 1.

78. The kit of any one of claims 71-77, wherein said single stranded nucleic acid is RNA or DNA.

79. The kit of any one of claims 71-78, wherein said reduced rate comprises a 10% or more reduction in cleavage.

80. The kit of any one of claims 71-79, wherein the kit further comprises labeled single stranded nucleic acid for detection.

81. The kit of any one of claims 71-78, wherein the kit further comprises crRNA comprising a sequence which recognizes target nucleic acid.

82. A kit comprising a Cas12a2 molecule, wherein said Cas12a2 molecule has been modified such that it indiscriminately cleaves single stranded DNA upon recognition of a specific complementary RNA target, and further wherein one or more residues are mutated such that double stranded nucleic acid and single stranded RNA is cleaved at a reduced rate compared to native Cas12a2.

83. The kit of claim 82, wherein Cas12a2 is represented by a protein with 80% or more identity to SEQ ID NO: 1.

84. The kit of claim 82 or 83, wherein residue Y1069 is mutated.

85. The kit of claim 82 or 83, wherein said mutation comprises a Y1069A substitution.

86. The kit of any one of claims 83-85, wherein said protein is 90% or more identical to SEQ ID NO: 1.

87. The kit of any one of claims 83-86, wherein said protein is 95% or more identical to SEQ ID NO: 1.

88. The kit of any one of claims 82-87, wherein said reduced rate comprises a 10% or more reduction in cleavage.

89. The kit of any one of claims 82-88, wherein the kit further comprises labeled single stranded nucleic acid for detection.

90. The kit of any one of claims 82-89, wherein the kit further comprises crRNA comprising a sequence which recognizes target nucleic acid.
